(19) United States
(12) Patent Application Publication
(10) Pub. No.: US 2025/0235642 A1
(43) Pub. Date: Jul. 24, 2025

(54) NASAL DELIVERY DEVICES

(71) Applicant: OptiNose, Inc., Yardley, PA (US)

(72) Inventors: Per Gisle DJUPESLAND, Oslo (NO); Joseph GORDON, Mansfield, MA (US); Michael LECLERC, Cranston, RI (US); Ramy A MAHMOUD, Skillman, NJ (US); Shane SIWINSKI, Barrington, RI (US)

(21) Appl. No.: 19/027,278

(22) Filed: Jan. 17, 2025

Related U.S. Application Data
(63) Continuation of application No. 17/305,269, filed on Jul. 2, 2021, now abandoned.
Application No. 17/305,269 is a continuation of application No. 16/102,898, filed on Aug. 14, 2018, now abandoned.
Application No. 16/102,898 is a continuation of application No. 14/380,801, filed on Aug. 25, 2014, now Pat. No. 10,076,614.
(60) Provisional application No. 61/603,093, filed on Feb. 24, 2012.

(51) Int. Cl. A61M 15/08; A61M 15/00

(52) U.S. Cl. CPC A61M 15/08; A61M 15/0021; A61M 15/0098; A61M 2202/064; A61M 2205/0216; A61M 2210/0618; A61M 2210/0625

(57) ABSTRACT

A nasal delivery device for delivering substance to a nasal cavity of a subject comprises: a housing (15); a nosepiece (17) for fitting to a nasal cavity of the subject; a mouthpiece (19) through which the subject in use exhales; and a flexible coupling (20) which couples the mouthpiece to the housing, wherein the flexible coupling provides for asymmetric translation of the mouthpiece relative to the nosepiece.

The present invention relates to a nasal delivery device for and a method of delivering substances, in particular one of a liquid, as a suspension or solution, or a powder, containing a medicament, especially systemic or topical pharmaceuticals, or a vaccine, to the nasal airway of a subject.

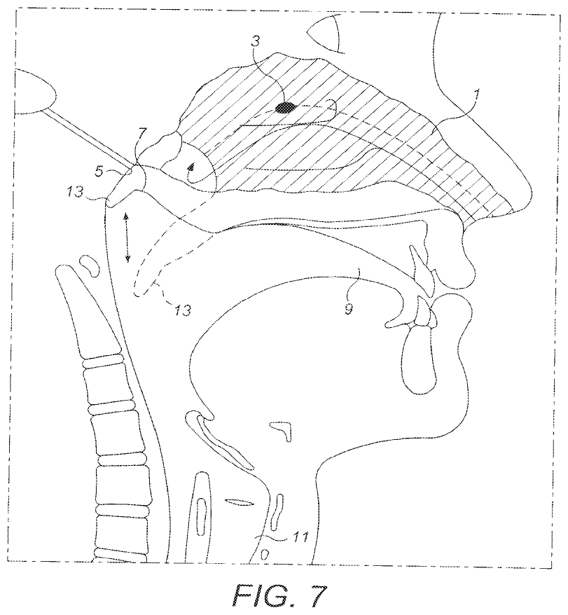

Referring to FIG. 7, the nasal airway 1 comprises the two nasal cavities separated by the nasal septum, which airway 1 includes numerous ostia, such as the paranasal sinus ostia 3 and the tubal ostia 5, and olfactory cells, and is lined by the nasal mucosa. The nasal airway 1 can communicate with the nasopharynx 7, the oral cavity 9 and the lower airway 11, with the nasal airway 1 being in selective communication with the anterior region of the nasopharynx 7 and the oral cavity 9 by opening and closing of the oropharyngeal velum 13. The velum 13, which is often referred to as the soft palate, is illustrated in solid line in the closed position, as achieved by providing a certain positive pressure in the oral cavity 9, such as achieved on exhalation through the oral cavity 9, and in dashed line in the open position.

There are many nasal conditions which require treatment. One such condition is nasal inflammation, specifically rhinitis, which can be allergic or non-allergic and is often associated with infection and prevents normal nasal function. By way of example, allergic and non-allergic inflammation of the nasal airway can typically effect between 10 and 20% of the population, with nasal congestion of the erectile tissues of the nasal concha, lacrimation, secretion of watery mucus, sneezing and itching being the most common symptoms. As will be understood, nasal congestion impedes nasal breathing and promotes oral breathing, leading to snoring and sleep disturbance. Other nasal conditions include nasal polyps which arise from the paranasal sinuses, hypertrophic adenoids, secretory otitis media, sinus disease and reduced olfaction.

In the treatment of certain nasal conditions, the topical administration of medicaments is preferable, particularly where the nasal mucosa is the prime pathological pathway, such as in treating or relieving nasal congestion. Medicaments that are commonly topically delivered include decongestants, anti-histamines, cromoglycates, steroids and antibiotics. At present, among the known anti-inflammatory pharmaceuticals, topical steroids have been shown to have an effect on nasal congestion. Topical decongestants have also been suggested for use in relieving nasal congestion. The treatment of hypertrophic adenoids and chronic secretory otitis media using topical decongestants, steroids and anti-microbial agents, although somewhat controversial, has also been proposed. Further, the topical administration of pharmaceuticals has been used to treat or at least relieve symptoms of inflammation in the anterior region of the nasopharynx, the paranasal sinuses and the auditory tubes.

Medicaments can also be systemically delivered through the nasal pathway, the nasal pathway offering a good administration route for the systemic delivery of pharmaceuticals, such as hormones, for example, oxytocin and calcitonin, and analgetics, such as anti-migraine compositions, as the high blood flow and large surface area of the nasal mucosa advantageously provides for rapid systemic uptake.

Nasal delivery is also expected to be advantageous for the administration of medicaments requiring a rapid onset of action, for example, analgetics, anti-emetics, insulin, anti-epileptics, sedatives and hypnotica, and also other pharmaceuticals, for example, cardio-vascular drugs. It is envisaged that nasal administration will provide for a fast onset of action, at a rate similar to that of injection and at a rate much faster than that of oral administration. Indeed, for the treatment of many acute conditions, nasal administration is advantageous over oral administration, since gastric stasis can further slow the onset of action following oral administration.

It is also expected that nasal delivery could provide an effective delivery route for the administration of proteins and peptides as produced by modern biotechnological techniques. For such substances, the metabolism in the intestines and the first-pass-effect in the liver represent significant obstacles for reliable and cost-efficient delivery.

Furthermore, it is expected that nasal delivery using the nasal delivery technique of the present invention will prove effective in the treatment of many common neurological diseases, such as Alzheimer's, Parkinson's, psychiatric diseases and intracerebral infections, where not possible using existing techniques. The nasal delivery technique of the present invention allows for delivery to the olfactory region, which region is located in the superior region of the nasal cavities and represents the only region where it is possible to circumvent the blood-to-brain barrier (BBB) and enable communication with the cerebrospinal fluid (CSF) and the brain.

Also, it is expected that the nasal delivery technique of the present invention will allow for the effective delivery of vaccines.

Aside from the delivery of medicaments, the irrigation of the nasal mucosa with liquids, in particular saline solutions, is commonly practised to remove particles and secretions, as well as to improve the mucociliary activity of the nasal mucosa. These solutions can be used in combination with active pharmaceuticals.

For any kind of drug delivery, accurate and reliable dosing is essential, but it is of particular importance in relation to the administration of potent drugs which have a narrow therapeutic window, drugs with potentially serious adverse effects and drugs for the treatment of serious and life-threatening conditions. For some conditions, it is essential to individualize the dosage to the particular situation, for example, in the case of diabetes mellitus. For diabetes, and, indeed, for many other conditions, the dosage of the pharmaceutical is preferably based on actual real-time measurements.

Currently, blood samples are most frequently used, but the analysis of molecules in the exhalation breath of subjects has been proposed as an alternative to blood analysis for several conditions. Breath analysis is currently used for the diagnosis of conditions such as Helicobacter pylori infections which cause gastric ulcers.

WO-A-2000/051672 discloses a delivery device for delivering a substance, in particular a medicament, in a bi-directional flow through the nasal cavities, that is, an air flow which passes into one nostril, around the posterior margin of the nasal septum and in the opposite direction out of the other nostril. This bi-directional air flow advantageously acts to stimulate the sensory nerves in the nasal mucosa, thereby conditioning the subject for the delivery and providing a more comfortable delivery situation.

It is an aim of the present invention to provide nasal delivery devices and nasal delivery methods for providing for delivery of a substance to a nasal cavity of subject, and in particular relatively-simple mechanically-actuatable delivery devices.

In one aspect the present invention provides a nasal delivery device for delivering substance to a nasal cavity of a subject, the delivery device comprising: a housing; a nosepiece for fitting to a nasal cavity of the subject; a mouthpiece through which the subject in use exhales; and a flexible coupling which couples the mouthpiece to the housing, wherein the flexible coupling provides for asymmetric translation of the mouthpiece relative to the nosepiece.

In one embodiment the nosepiece includes an outlet from which substance is delivered, and a seat against which the nare of a nostril of the subject is in use seated, in achieving a sealing fit between the nosepiece and the nasal cavity of the subject.

In one embodiment the nosepiece includes a tapered section which in use is located within a nasal cavity of the subject and tapers outwardly from the outlet.

In one embodiment the mouthpiece comprises a tubular section.

In one embodiment the tubular section is formed of a rigid material.

In one embodiment the tubular section is formed of a semi-rigid material.

In one embodiment the flexible coupling is a resilient element.

In one embodiment the asymmetric translation of the mouthpiece relative to the nosepiece provides for greater movement in a direction along the axis of the nosepiece than in a direction laterally to the nosepiece.

In one embodiment the distal end of the mouthpiece is configured to move a distance at least 1.5 times greater in a direction parallel to the axis of the nosepiece than in a direction orthogonally to the axis of the nosepiece.

In one embodiment the distal end of the mouthpiece is configured to move a distance at least 1.75 times greater in a direction parallel to the axis of the nosepiece than in a direction orthogonally to the axis of the nosepiece.

In one embodiment the distal end of the mouthpiece is configured to move a distance at least 2 times greater in a direction parallel to the axis of the nosepiece than in a direction orthogonally to the axis of the nosepiece.

In one embodiment the flexible coupling comprises an annular coupling member which is attached in one part to the housing and another part to the mouthpiece, such that exhalation through the mouthpiece delivers an air flow into the housing.

In one embodiment the coupling member is configured to provide a hinge section about which the mouthpiece is preferentially hinged when biased upwardly or downwardly by application of a biasing force.

In one embodiment the hinge section is provided to one side thereof, proximate the nosepiece.

In one embodiment the coupling member has a shorter dimension to the one side thereof, thereby ensuring that the mouthpiece is hinged about the one side of the coupling member.

In one embodiment the coupling member has a progressively-increasing dimension to the other side thereof, distal the nosepiece.

In one embodiment the coupling member has an arcuate, bowed profile which becomes larger towards the other side thereof, and provides for stretching when the mouthpiece is biased upwardly and compression when the mouthpiece is biased downwardly.

In one embodiment the profile section is bowed outwardly, whereby the biasing force required to bias the mouthpiece upwardly is less than the biasing force required to bias the mouthpiece downwardly.

In one embodiment the profile section of the coupling member is formed of graded material, such that the material of the coupling member is less resilient at the one side thereof than the other side thereof.

In one embodiment the coupling member is formed of graded material, such that the material of the coupling member is less resilient at the one side thereof than the other side thereof.

In one embodiment the coupling member is configured to provide the axis of the mouthpiece at an angle of between about 45 and about 55 degrees relative to the axis of the nosepiece.

In one embodiment the coupling member is configured to provide the axis of the mouthpiece at an angle of between about 48 and about 52 degrees relative to the axis of the nosepiece.

In one embodiment the coupling member is configured to provide the axis of the mouthpiece at an angle of about 50 degrees relative to the axis of the nosepiece.

In one embodiment the coupling member is configured to allow for the mouthpiece to be moved upwardly through an angle of between about 7 and about 17 degrees relative to the axis of the nosepiece.

In one embodiment the coupling member is configured to allow for the mouthpiece to be moved upwardly through an angle of between about 9 and about 15 degrees relative to the axis of the nosepiece.

In one embodiment the coupling member is configured to allow for the mouthpiece to be moved upwardly through an angle of between about 10 and about 14 degrees relative to the axis of the nosepiece.

In one embodiment the coupling member is configured to allow for the mouthpiece to be moved upwardly through an angle of about 12 degrees relative to the axis of the nosepiece.

In one embodiment the coupling member is configured to allow for the mouthpiece to be moved downwardly through an angle of between about 4 and about 10 degrees relative to the axis of the nosepiece.

In one embodiment the coupling member is configured to allow for the mouthpiece to be moved downwardly through an angle of between about 5 and about 9 degrees relative to the axis of the nosepiece.

In one embodiment the coupling member is configured to allow for the mouthpiece to be moved downwardly through an angle of between about 6 and about 8 degrees relative to the axis of the nosepiece.

In one embodiment the coupling member is configured to allow for the mouthpiece to be moved downwardly through an angle of about 7 degrees relative to the axis of the nosepiece.

In one embodiment the coupling member is formed of a thermoplastic elastomer (TPE).

In one embodiment the TPE has a durometer of between about 40 and about 60.

In one embodiment the TPE has a durometer of between about 45 and about 55.

In one embodiment the TPE has a durometer of about 50.

In one embodiment the delivery device further comprises: a substance supply unit which is manually actuated to deliver substance to the nasal cavity of the subject.

In another aspect the present invention provides a nasal delivery device for delivering substance to a nasal cavity of a subject, the delivery device comprising: a housing; a nosepiece for fitting to a nasal cavity of the subject; a mouthpiece through which the subject in use exhales; and a flexible coupling which couples the mouthpiece to the housing, providing for movement of the mouthpiece relative to the nosepiece.

In a further aspect the present invention provides a method of delivering substance to a nasal cavity of a subject, the method comprising the steps of: providing a nasal delivery device comprising: a housing; a nosepiece for fitting to a nasal cavity of the subject; a mouthpiece through which the subject in use exhales; and a flexible coupling which couples the mouthpiece to the housing, wherein the flexible coupling provides for asymmetric translation of the mouthpiece relative to the nosepiece; fitting the nosepiece to a nasal cavity of the subject; and locating the mouthpiece in the mouth of the subject by flexing the asymmetric coupling and thereby asymmetrically translating the mouthpiece relative to the nosepiece.

Preferred embodiments of the present invention will now be described hereinbelow by way of example only with reference to the accompanying drawings, in which:

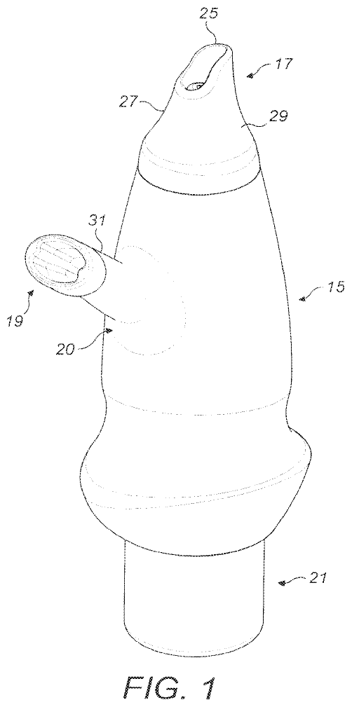

FIG. 1 illustrates a perspective view of a nasal delivery device in accordance with a first embodiment of the present invention;

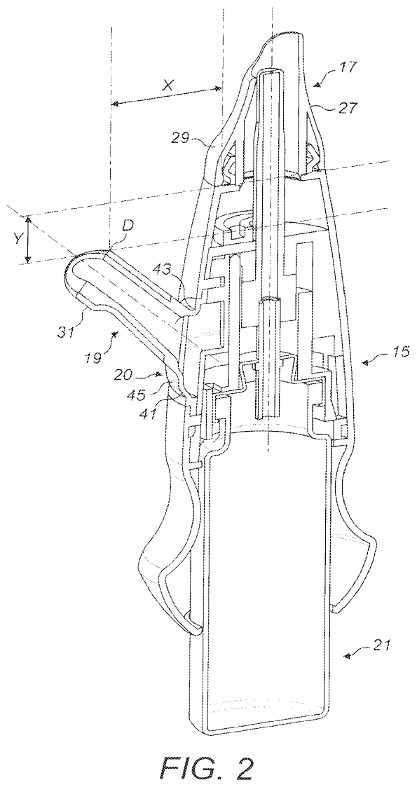

FIG. 2 illustrates a vertical sectional view of the delivery device of FIG. 1, with the mouthpiece in the at rest position;

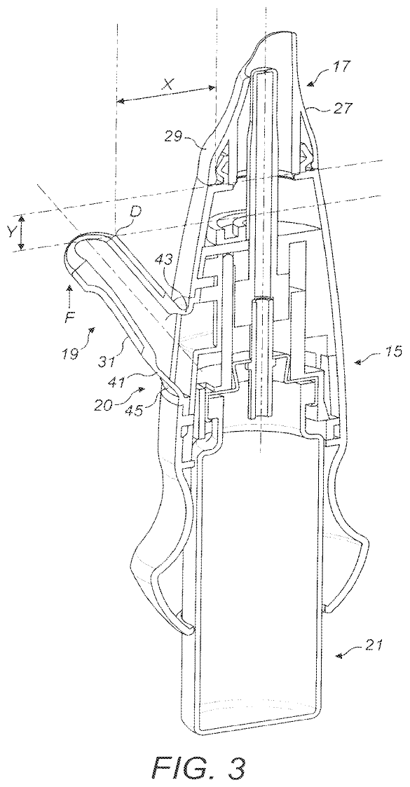

FIG. 3 illustrates a vertical sectional view of the delivery device of FIG. 1, with the mouthpiece in a position biased upwardly relative to the nosepiece;

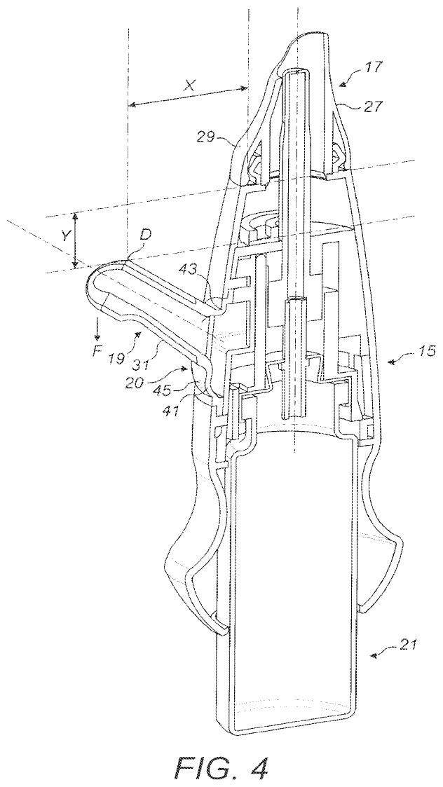

FIG. 4 illustrates a vertical sectional view of the delivery device of FIG. 1, with the mouthpiece in a position biased downwardly relative to the nosepiece;

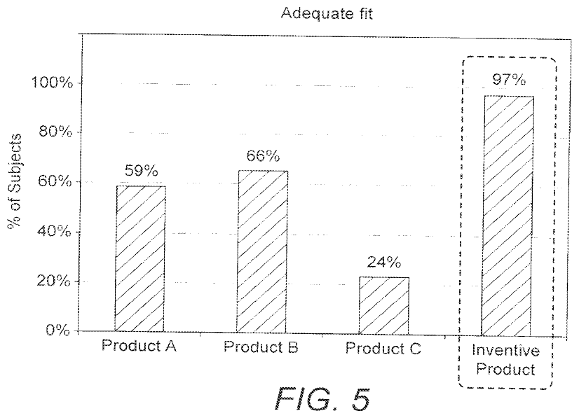

FIG. 5 illustrates the results of a study to determine adequacy of fit of the delivery device of FIG. 1, as compared to three comparator devices;

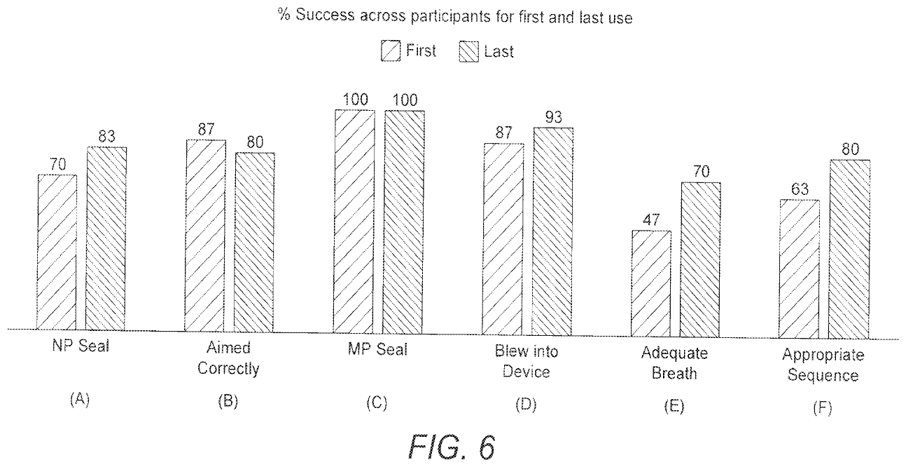

FIG. 6 illustrates success of subjects in achieving a plurality of steps in fitting the delivery device of FIG. 1; and

FIG. 7 schematically illustrates the anatomy of the upper respiratory tract of a human subject.

The delivery device comprises a housing 15, a nosepiece 17 for fitting in a nasal cavity of a subject, a mouthpiece 19 through which the subject in use exhales, a flexible coupling 20 which couples the mouthpiece 19 to the housing 15, and a substance supply unit 21 which is manually actuated to deliver substance to the nasal cavity of the subject.

The nosepiece 17 includes an outlet 25 from which substance is delivered, a tapered section 27 which in use is located within a nasal cavity of the user and tapers outwardly from the outlet 25, and a seat 29 against which the nare of the nostril is in use seated, in achieving a sealing fit between the nosepiece 17 and the nasal cavity of the user.

The mouthpiece 19 comprises a tubular section 31, in this embodiment of a rigid or semi-rigid material.

The flexible coupling 20 is a resilient element which allows for movement of the mouthpiece 19 relative to the nosepiece 17, in this embodiment an asymmetric translation of the mouthpiece 19 relative to the nosepiece 17.

The present inventors have recognized that a fixed relationship between the mouthpiece 19 and the nosepiece 17 would not allow the delivery device to accommodate sufficient of the possible patient population using a single delivery device, given the variance that exists between patients, particularly in terms of age, gender and ethnicity, and have further recognized that an entirely free and flexible coupling between the mouthpiece 19 and the nosepiece 17, without any constraint, would not be sufficient, in not maintaining a desired relationship between the mouthpiece 19 and the nosepiece 17, which the present inventors have determined to be necessary to achieve a required orientation of the delivery device for optimizing delivery of substance.

The present inventors have determined that the provision of asymmetric translation of the mouthpiece 19 relative to the nosepiece 17 when the mouthpiece 19 is moved, and specifically in a manner which provides for greater movement in a direction along the axis of the nosepiece 17 than in a direction laterally to the nosepiece 17, provides an arrangement which allows for fitting of a single-sized delivery device in a much greater range of the possible patient population.

FIG. 5 illustrates the results of a study of 29 subjects to determine adequacy of fit of the delivery device, as compared to three comparator delivery devices (Products A, B and C). Adequate fit is defined as the delivery device fitting the subject and sealing sufficiently in the nose and mouth as to allow use of the delivery device, though may not find the delivery device preferable or comfortable.

In this study, the asymmetric translation of the mouthpiece 19 relative to the nosepiece 17 provides an arrangement which allowed for fitting of a single-sized delivery device in 28 of 29 subjects.

FIG. 6 illustrates success of subjects in achieving a plurality of steps in fitting the delivery device.

In this study, the subjects were required repeatedly to perform the steps of (1) fitting the nosepiece 17 in a nasal cavity, (2) locating the mouthpiece 19 in the mouth, and (3) blowing into the mouthpiece 19.

In these steps, the following parameters were measured for the first and last sequence of steps: (A) achieving a proper seal at the nosepiece 17, (B) aiming the nosepiece 17 correctly, (C) achieving a proper seal at the mouthpiece 19, (D) blowing into the mouthpiece 19, (E) blowing into the mouthpiece 19 with adequate exhalation force, and (F) performing the sequence of steps in the appropriate order.

As will be observed, a very high degree of patient compliance is achieved by the delivery device, which improves with use of the delivery device.

In this embodiment the distal end D of the mouthpiece 19 is configured to move a distance Y at least 1.5 times greater in a direction parallel to the axis of the nosepiece 17 than in a direction X orthogonally to the axis of the nosepiece 17. More preferably, the distal end D of the mouthpiece 19 is configured to move a distance at least 1.75 times or at least 2 times greater in a direction Y parallel to the axis of the nosepiece 17 than in a direction X orthogonally to the axis of the nosepiece 17.

In this embodiment the flexible coupling 20 comprises an annular coupling member 41 which is attached in one part to the housing 15 and another part to the tubular section 31 of the mouthpiece 19, such that exhalation through the mouthpiece 19 delivers an air flow into the housing 15.

In this embodiment the coupling member 41 is configured to provide a hinge section 43, here, to one, upper side thereof, proximate the nosepiece 17, about which the mouthpiece 19 is preferentially hinged when biased upwardly or downwardly by the application of a biasing force F.

In this embodiment the coupling member 41 has a shorter dimension to the one, upper side, thereby ensuring that the mouthpiece 19 is hinged about the one, upper side, and a progressively-increasing dimension to the other, lower side, distal the nosepiece 17.

In this embodiment the coupling member 41 has an arcuate, bowed profile 45 which becomes larger towards the other lower side, and allows for stretching in the event of the mouthpiece 19 being biased upwardly, as illustrated in FIG. 3, and compression in the event of the mouthpiece 19 being biased downwardly, as illustrated in FIG. 4.

In this embodiment the profile section 45 is bowed outwardly, whereby the biasing force required to bias the mouthpiece 19 upwardly is less than the biasing force required to bias the mouthpiece 19 downwardly. Again, for reasons of optimizing fitting and orientation of the delivery device, the present inventors have recognized that this is achieved by requiring the mouthpiece 19 to be biased upwardly from a lower position. Thus, the delivery device is configured to facilitate operation by providing that raising the mouthpiece 19 is easier than lowering the mouthpiece 19, and this is further promoted by configuring the mouthpiece 19 such that the position of the mouthpiece 19 is lower than required for a majority of the patient population.

In this embodiment the coupling member 41 is configured to provide the axis of the mouthpiece 19 at an angle of about 50 degrees relative to the axis of the nosepiece 17, and allow for the mouthpiece 19 to be moved upwardly through an angle of about 12 degrees to enclose an angle of about 38 degrees relative to the axis of the nosepiece 17 and downwardly through an angle of about 7 degrees to enclose an angle of about 57 degrees relative to the axis of the nosepiece 17.

In an alternative embodiment the coupling member 41, instead or in addition to having a bowed profile section 45, can be formed of graded material, such that the material of the coupling member 41 is less resilient at the one, upper side than the other, lower side.

In this embodiment the coupling member 41 is formed of a thermoplastic elastomer (TPE), preferably having a durometer of 50.

Finally, it will be understood that the present invention has been described in its preferred embodiments and can be modified in many different ways without departing from the scope of the invention as defined by the appended claims.

1-38. (canceled)

39. A method of delivering a pharmaceutical substance to a nasal cavity of a subject, the method comprising:
providing a delivery device comprising:
a housing,
a nosepiece for fitting to a nostril of the subject, the nosepiece having a longitudinal axis and including an outlet from which the pharmaceutical substance is in use delivered to the nasal cavity of the subject,
a mouthpiece for fitting to a mouth of the subject and into which the subject in use exhales, the mouthpiece including a tubular section and having a longitudinal axis,
a flexible coupling connecting the mouthpiece to the housing, wherein the flexible coupling comprises an annular coupling member which is attached at a first end to the housing and at a second end to the tubular section of the mouthpiece, wherein the mouthpiece is configured to move towards or away from the longitudinal axis of the nosepiece when biased towards or away from the nosepiece by application of respective manually-applied biasing forces and a first side of the annular coupling member proximate the nosepiece is relatively shorter in length than a second side of the annular coupling member distal the nosepiece to form an acute angle between the longitudinal axis of the nosepiece and the longitudinal axis of the mouthpiece, and
a substance delivery system configured to deliver the pharmaceutical substance through the outlet of the nosepiece;
wherein a manually-applied biasing force required to move the tubular section of the mouthpiece towards the longitudinal axis of the nosepiece by biasing the tubular section of the mouthpiece towards the nosepiece is lower than a manually-applied biasing force required to move the tubular section of the mouthpiece away from the longitudinal axis of the nosepiece by biasing the tubular section of the mouthpiece away from the nosepiece, and
wherein the annular coupling member has an arcuate, bowed structure on the second side thereof that becomes progressively larger towards the second side and is configured to stretch when the tubular section of the mouthpiece is biased towards the nosepiece and compress when the tubular section of the mouthpiece is biased away from the nosepiece;
causing the tubular section of the mouthpiece to be biased towards the nosepiece in fitting the nosepiece to the nostril of the subject and the mouthpiece to the mouth of the subject; and
causing the substance delivery system to be actuated to deliver the pharmaceutical substance through the outlet of the nosepiece while the subject is exhaling through the mouthpiece and an exhalation air flow is delivered through the nosepiece.

40. The method of claim 39, wherein the nosepiece includes a seat against which a nare of the nostril of the subject is in use seated to achieve a sealing fit between the nosepiece and the nare of the subject.

41. The method of claim 39, wherein the longitudinal axis of the mouthpiece is disposed at an angle of between about 45 and about 55 degrees relative to the longitudinal axis of the nosepiece.

42. The method of claim 41, wherein the longitudinal axis of the mouthpiece is disposed at an angle of between about 48 and about 52 degrees relative to the longitudinal axis of the nosepiece.

43. The method of claim 41, wherein the annular coupling member is configured to permit movement of the tubular section of the mouthpiece towards the nosepiece through an angle of between about 7 and about 17 degrees relative to the longitudinal axis of the nosepiece.

44. The method of claim 43, wherein the annular coupling member is configured to permit movement of the tubular section of the mouthpiece towards the nosepiece through an angle of between about 9 and about 15 degrees relative to the longitudinal axis of the nosepiece.

45. The method of claim 43, wherein the annular coupling member is configured to permit movement of the tubular section of the mouthpiece towards the nosepiece through an angle of between about 10 and about 14 degrees relative to the longitudinal axis of the nosepiece.

46. The method of claim 41, wherein the annular coupling member is configured to permit movement of the tubular section of the mouthpiece away from the nosepiece through an angle of between about 4 and about 10 degrees relative to the longitudinal axis of the nosepiece.

47. The method of claim 46, wherein the annular coupling member is configured to permit movement of the tubular section of the mouthpiece away from the nosepiece through an angle of between about 5 and about 9 degrees relative to the longitudinal axis of the nosepiece.

48. The method of claim 46, wherein the annular coupling member is configured to permit movement of the tubular section of the mouthpiece away from the nosepiece through an angle of between about 6 and about 8 degrees relative to the longitudinal axis of the nosepiece.

49. The method of claim 39, wherein the annular coupling member is formed of a thermoplastic elastomer (TPE).

50. The method of claim 49, wherein the TPE has a durometer of between about 40 and about 60.

51. The method of claim 50, wherein the TPE has a durometer of between about 45 and about 55.

52. The method of claim 39, wherein the second side of the annular coupling member forms an obtuse angle relative to the housing.

53. The method of claim 39, wherein the pharmaceutical substance is a liquid substance.

54. The method of claim 39, wherein the pharmaceutical substance is a topical substance.

55. The method of claim 39, wherein the pharmaceutical substance is a steroid.

56. The method of claim 39, wherein the pharmaceutical substance is a powder substance.

57. The method of claim 39, wherein the pharmaceutical substance is a liquid topical steroid substance.

58. A method of delivering a liquid steroid substance to a nasal cavity of a subject, the method comprising:
providing a delivery device comprising:
a housing,
a nosepiece for fitting to a nostril of the subject, the nosepiece having a longitudinal axis and including an outlet from which the liquid steroid substance is in use delivered to the nasal cavity of the subject,
a mouthpiece for fitting to a mouth of the subject and into which the subject in use exhales, the mouthpiece including a tubular section and having a longitudinal axis,
a flexible coupling connecting the mouthpiece to the housing, wherein the flexible coupling comprises an annular coupling member which is attached at a first end to the housing and at a second end to the tubular section of the mouthpiece, wherein the mouthpiece is configured to move towards or away from the longitudinal axis of the nosepiece when biased towards or away from the nosepiece by application of respective manually-applied biasing forces, and a first side of the annular coupling member proximate the nosepiece is relatively shorter in length than a second side of the annular coupling member distal the nosepiece to form an acute angle between the longitudinal axis of the nosepiece and the longitudinal axis of the mouthpiece, and
a manually-actuated substance delivery system configured to deliver the liquid steroid substance through the outlet of the nosepiece during manual actuation of the manually-actuated substance delivery system;
wherein a manually-applied biasing force required to move the tubular section of the mouthpiece towards the longitudinal axis of the nosepiece by biasing the tubular section of the mouthpiece towards the nosepiece is lower than a manually-applied biasing force required to move the tubular section of the mouthpiece away from the longitudinal axis of the nosepiece by biasing the tubular section of the mouthpiece away from the nosepiece, and
wherein the annular coupling member has an arcuate, bowed structure on the second side thereof that becomes progressively larger towards the second side and is configured to stretch when the tubular section of the mouthpiece is biased towards the nosepiece and compress when the tubular section of the mouthpiece is biased away from the nosepiece;
causing the tubular section of the mouthpiece to be biased towards the nosepiece in fitting the nosepiece to the nostril of the subject and the mouthpiece to the mouth of the subject; and
causing the substance delivery system to be actuated to deliver the liquid steroid substance through the outlet of the nosepiece while the subject is exhaling through the mouthpiece and an exhalation air flow is delivered through the nosepiece.
